(19) United States
(12) Patent Application Publication
(10) Pub. No.: US 2013/0266629 A1
(43) Pub. Date: Oct. 10, 2013

(54) MEDICAL DEVICE HAVING A SURFACE COMPRISING ANTIMICROBIAL METAL

(71) Applicant: Anna ARVIDSSON, Goteborg (SE)

(72) Inventor: Anna ARVIDSSON, Goteborg (SE)

(73) Assignee: DENTSPLY INTERNATIONAL INC., York, PA (US)

(21) Appl. No.: 13/798,447

(22) Filed: Mar. 13, 2013

Related U.S. Application Data
(60) Provisional application No. 61/617,940, filed on Mar. 30, 2012.

(30) Foreign Application Priority Data
Mar. 30, 2012 (EP) 12162632.9

(51) Int. Cl. A61L 31/08

(52) U.S. Cl. CPC A61L 31/088
USPC 424/411; 424/409; 424/650; 424/653; 427/2.24

(57) ABSTRACT

A medical device intended for contact with living tissue comprises a substrate having a surface, which surface comprises a layer comprising one or more compound(s)) of at least one non-toxic post-transition metal, such as a gallium or bismuth compound. A layer comprising a compound of a non-toxic post-transition metal has been shown to inhibit biofilm formation on the surface of the medical device, which may reduce the risk for infection e.g. around a dental implant. A method of producing the medical device comprises: a) providing a substrate having a surface; and applying a compound of a non-toxic post-transition metal onto said surface to form a layer, e.g. using a thin film deposition technique.

### CROSS-REFERENCE TO RELATED APPLICATIONS

This patent application claims the benefit of and priority to EP Application Ser No. 12162632.9, filed on Mar. 30, 2012 and U.S. Provisional Patent Application Ser. No. 61/617,940, filed on Mar. 30, 2012, which are herein incorporated by reference for all purposes.

### FIELD OF THE INVENTION

The present invention relates to a medical device having a surface layer comprising an anti-microbial metal, and to methods of producing such a device.

### BACKGROUND OF THE INVENTION

For any type of medical device intended for contact with living tissue, biocompatibility is a crucial issue. The risk for foreign body reaction, clot formation and infection, among many other things, must be addressed and minimized in order to avoid adverse effects, local as well as systemic, which may otherwise compromise the health of the patient and/or lead to failure of the device. This is particularly the case for permanent implants.

Healing or regeneration of tissue around an implant is often vital in order to secure the implant and its long-term functionality. This is especially important for load-bearing implants such as dental or orthopedic implants.

Dental implant systems are widely used for replacing damaged or lost natural teeth. In such implant systems, a dental fixture (screw), usually made of titanium or a titanium alloy, is placed in the jawbone of the patient in order to replace the natural tooth root. An abutment structure is then attached to the fixture in order to build up a core for the part of the prosthetic tooth protruding from the bone tissue, through the soft gingival tissue and into the mouth of the patient. On said abutment, the prosthesis or crown may finally be seated.

For dental fixtures, a strong attachment between the bone tissue and the implant is necessary. For implants intended for contact with soft tissue, such as abutments which are to be partially located in the soft gingival tissue, also the compatibility with soft tissue is vital for total implant functionality. Typically, after implantation of a dental implant system, an abutment is partially or completely surrounded by gingival tissue. It is desirable that the gingival tissue should heal quickly and firmly around the implant, both for medical and aesthetic reasons. A tight sealing between the oral mucosa and the dental implant serves as a barrier against the oral microbial environment and is crucial for implant success. This is especially important for patients with poor oral hygiene and/or inadequate bone or mucosal quality. Poor healing or poor attachment between the soft tissue and the implant increases the risk for infection and perk implantitis, which may ultimately lead to bone resorption and failure of the implant.

There are several strategies for increasing the chances of a successful implantation of a medical device, for example enhancing the rate of new tissue formation and/or, in instances where tissue-implant bonding is desired, enhancing the rate of tissue attachment to the implant surface, or by reducing the risk for infection. Enhancement of new tissue formation may be achieved for example by various surface modifications and/or deposition of bioactive agents on the surface.

The risk of infection in connection with dental implants is today primarily addressed by preventive measures, such as maintaining good oral hygiene. Once a biofilm is formed on the surface of a dental implant, it is difficult to remove it by applying antibacterial agents. In the case of infection in the bone or soft tissue surrounding a dental implant (peri-implantitis), mechanical debridement is the basic element, sometimes in combination with antibiotics, antiseptics, and/or ultrasonic or laser treatment.

### SUMMARY OF THE INVENTION

It is an object of the present invention to overcome this problem, and to provide a medical device, such as an implant, having a surface, which reduces the risk for infection upon contact of the medical device with living tissue.

According to a first aspect of the invention, this and other objects are achieved by medical device intended for contact with living tissue, comprising a substrate having a surface layer comprising one or more compound(s) of a non-toxic post-transition metal.

The layer comprising one or more compound(s) of a non-toxic post-transition metal, e.g. gallium compound(s), may have an atomic concentration (at %) of post-transition metal, e.g. gallium and/or bismuth, of at least 5 at %. In embodiments of the invention, the gallium concentration in said layer is at least 10 at %, for example at least 15 at %, e.g. at least 20 at %. However in other embodiments of the invention, the content of the post-transition metal may be less than 5%, for example at least 0.05 at %. The layer may have a gallium content of up to 50 at %.

In embodiments of the invention, the compound of a non-toxic post-transition metal constitutes the major part of the layer.

A medical device surface having a layer incorporating a non-toxic post-transition metal such as gallium or bismuth has been shown to be effective against various bacterial strains, and was shown to inhibit biofilm formation in vitro. The medical device according to the invention may also be effective against other microbes, such as fungi.

In embodiments of the invention, said living tissue is soft tissue. Alternatively, said living tissue may be cartilage or bone tissue.

In embodiments of the invention, said one or more compound(s) of a non-toxic post-transition metal also comprises an additional metal, for example a biocompatible metal such as titanium. Titanium is known to be well tolerated by living tissue, and has been used as an implant material for many years. By including a biocompatible metal, e.g. titanium, in the gallium-containing layer, a surface is obtained which is more similar to established implant surfaces and which is even more likely to be well tolerated by living tissue.

In embodiments of the invention, the surface layer may be a metallic layer. The compound comprising a non-toxic post-transition metal may be a metallic compound.

In embodiments of the invention, the non-toxic post-transition metal is selected from bismuth and gallium. Hence, the compound(s) of the non-toxic post-transition metal may be selected from bismuth compound(s) and gallium compound(s).

A gallium compound may be selected from the group consisting of gallium-titanium oxide, gallium nitride, gallium-titanium nitride, gallium carbide, gallium selenide, and gallium sulphide, gallium chloride, gallium fluoride, gallium iodide, gallium oxalate, gallium phosphate, gallium maltolate, gallium acetate and gallium lactate. A bismuth compound may be selected from the group consisting of bismuth-titanium, bismuth-titanium oxide, bismuth-titanium nitride, bismuth nitride, bismuth carbide, bismuth selenide, and bismuth sulphide, bismuth chloride, bismuth fluoride, bismuth iodide, bismuth oxalate, bismuth phosphate, bismuth maltolate, bismuth acetate and bismuth lactate. These compounds of non-toxic post-transition metals are in general, well tolerated by living tissue of a mammal, and can be deposited on a surface using a thin film deposition technique.

In embodiments, the compound of a non-toxic post-transition metal is a nitride of at least one non-toxic post-transition metal, such as gallium nitride or bismuth nitride, optionally also comprising titanium. Nitrides containing non-toxic post-transition metals such as gallium or bismuth are particularly useful in the present invention, in particular for dental implant applications, because such compounds may provide an aesthetically desirable surface layer, in particular with respect to color. Such nitrides can be deposited using thin film deposition techniques. The nitride of a non-toxic post-transition metal may be selected from the group consisting of gallium-titanium nitride, gallium nitride, bismuth-titanium nitride, bismuth nitride, and gallium-bismuth-titanium nitride.

In embodiments of the invention, the layer comprising a compound of a non-toxic post-transition metal may further comprise a salt of a non-toxic post-transition metal, e.g. a gallium salt or a bismuth salt. The salt may be deposited onto the surface layer comprising the compound of a non-toxic post-transition metal. For example, a gallium salt or a bismuth salt may be deposited onto a first layer comprising a first gallium or bismuth compound. A salt deposit may increase the release of antimicrobial post-transition metal from the surface early after contact with living tissue, thus temporarily further enhancing an antibacterial or antimicrobial effect of the layer.

Generally, the layer comprising the compound of a post-transition metal may have a thickness in the range of from 10 nm to 1.5 μm. A layer of at least 10 nm may be sufficient to provide a desirable antibacterial effect, whereas thick layers of up to 1 μm may be desirable for aesthetic reasons, having a color suitable for e.g. dental implants.

Typically, in embodiments of the invention, the layer comprising the compound of a non-toxic post-transition metal may be a homogeneous layer. The layer may also be a non-porous layer. A non-porous layer is typically less susceptible of bacterial growth and biofilm formation compared to a porous layer.

The substrate on which the layer comprising the at least one gallium compound is provided may comprise a metallic material, typically a biocompatible metal or alloy such as titanium or titanium alloy. Alternatively, the substrate may comprise a ceramic material. In other embodiments, the substrate may comprise a polymeric material, or a composite material.

The medical device of the invention is typically an implant intended for long-term contact with, or implantation into, living tissue. In one embodiment the medical device is an implant intended for implantation at least partially into soft tissue. In yet other embodiments of the invention, the medical device may be intended for short-term or prolonged contact with living tissue, typically soft tissue.

For example, the medical device may be a dental implant, in particular a dental abutment. In another embodiment, the medical device may be a bone anchored hearing device. In another embodiment, the medical device may be an orthopaedic implant. In another embodiment, the medical device may be a stent. In another embodiment, the medical device may be a shunt. As another example, the medical device may be a catheter adapted for insertion into a bodily cavity such as a blood vessel, the digestive tract or the urinary system.

In another aspect, the invention provides a method of producing a medical device as described herein, comprising a) providing a substrate having a surface; and b) applying a compound of a non-toxic post-transition metal onto said surface to form a layer. Typically, the compound of a non-toxic post-transition metal is a nitride of a non-toxic post-transition metal, such as a nitride of gallium and/or bismuth.

In some embodiments, the application step b) involves simultaneously or sequentially applying a non-toxic post-transition metal and an additional metal or metal compound onto said surface. The additional metal or metal compound may comprise titanium. Applying the post-transition metal, and optionally also an additional metal or metal compound, can be achieved using a thin film deposition technique.

A medical device as described above may be used for preventing biofilm formation and/or bacterial infection of a surrounding tissue, in particular soft tissue. In particular the medical device of the invention may be used for preventing bacterial infection of gingival tissue and/or periimplantitis.

It is noted that the invention relates to all possible combinations of features recited in the claims.

### BRIEF DESCRIPTION OF THE DRAWINGS

FIG. 1 is a side view of a medical device according to an embodiment of the invention, wherein the medical device is a dental abutment.

FIG. 2 illustrates in cross-section part of a medical device according to embodiments of the invention, showing a substrate material and a layer comprising a gallium compound.

### DETAILED DESCRIPTION OF THE INVENTION

The present inventor has found that a medical device having a surface layer comprising a compound of a post-transition metal, in particular a non-toxic, antimicrobial post-transition metal, such as gallium and/or bismuth, provides very advantageous effects in terms of reduced risk of infection, improved tissue healing and/or aesthetic performance. The inventor has demonstrated that a titanium body having a surface coating incorporating gallium (Ga) can prevent the growth of bacteria on and around the surface and thus may be useful in preventing detrimental infection around e.g. a dental abutment implanted into the gingiva. It has also been demonstrated that a titanium body having a surface coating incorporating bismuth (Bi) can prevent the growth of bacteria on and around the surface and thus may be useful in preventing detrimental infection around e.g. a dental abutment implanted into the gingiva.

According to the present invention, a tissue contact surface of a surface of a medical device comprises a compound of a post-transition metal. Typically, the post-transition metal is non-toxic to mammalian cells at the concentrations that have a lethal effect on bacterial cells.

The term “post-transition metal” generally refers to metal elements found in groups 13-16 and periods 3-6 of the periodic table. Usually, aluminium, gallium, indium, thallium, tin, lead, bismuth and polonium are regarded as post-transition metals. In contrast, transition metals are formed of group 3-12 elements. Germanium and antimony are considered not as post-transition metals, but metalloid elements.

Group 16 of the periodic table only contains one post-transition metal: polonium, which is toxic. Hence, in the present invention, post-transition metals of groups 13-15 and periods 3-6 are preferred.

In embodiments of the invention, the post-transition metal used is non-toxic.

As used herein, “non-toxic” means that the substance (e.g. a compound or element) in question does not damage mammalian cells at concentrations that have a lethal effect on bacterial cells.

Examples of post-transition metals that are toxic include thallium (TI), lead (Pb) and polonium (Po). Other post-transitional metals (e.g. indium (In) and tin (Sn)) may be considered non-toxic in the pure metal form, but may be toxic in other forms or when they form compounds with other elements.

The non-toxic post transition metal used in the present invention is typically non-toxic when present in elementary form, as metal ions, and/or as one of the exemplary compounds given herein.

Furthermore, the post-transition metal used in the present invention typically has an antimicrobial or antibacterial effect. Post-transition metals that are considered to have some antimicrobial or antibacterial effect (including any oligodynamic effect) include at least gallium, tin, lead, and bismuth.

In view of the above, the invention may employ at least one non-toxic, antimicrobial post-transition metal, which may be selected from gallium and bismuth.

For example, a gallium compound may be applied to a medical device as a surface layer. Alternatively, a gallium compound could be incorporated into at least part of a body forming a medical device, such that at least one surface of the device comprises the gallium compound.

As another example, a bismuth compound may be applied to a medical device as a surface layer. Alternatively, a bismuth compound could be incorporated into at least part of a body forming a medical device, such that at least one surface of the device comprises the bismuth compound.

Gallium has been used in medicine at least since the 1940's, primarily as a radioactive agent for medical imaging. The antibacterial properties of gallium have been investigated in several studies. In Kaneko et al. (2007) it was established that gallium nitrate (Ga(NO₃)₃) inhibits growth of Pseudomonas aeruginosa in batch cultures. Olakanmi et al (2010) found that Ga(NO₃)₃ inhibited the growth of Francisella novicida. Gallium acts by disrupting iron metabolism. It may be assumed that gallium is also effective against other microbes, e.g. fungi such as yeasts or moulds.

Bismuth is known to possess antibacterial activity. Bismuth compounds were formerly used to treat syphilis, and bismuth subsalicylate and bismuth subcitrate are currently used to treat peptic ulcers caused by Helicobacter pylori. The mechanism of action of this substance is still not well understood. Bibrocathol is an organic bismuth-containing compound used to treat eye infections, and bismuth subsalicylate and bismuth subcarbonate are used as ingredients in antidiarrheal pharmaceuticals.

Directive 2007/47/ec defines a medical device as: “any instrument, apparatus, appliance, software, material or other article, whether used alone or in combination, including the software intended by its manufacturer to be used specifically for diagnostic and/or therapeutic purposes and necessary for its proper application, intended by the manufacturer to be used for human beings”. In the context of the present invention, only medical devices intended for contact with living tissue are considered, that is, any instrument, apparatus appliance, material or other article of physical character that is intended to be applied on, inserted into, implanted in or otherwise brought into contact with the body, a body part or an organ. Furthermore, said body, body part or organ may be that of a human or animal, typically mammal, subject. Preferably however the medical device is intended for human subjects. Medical devices included within the above definition are for example implants, catheters, shunts, tubes, stents, intrauterine devices, and prostheses.

In particular, the medical device may be a medical device intended for implantation into living tissue or for insertion into the body or a body part of a subject, including insertion into a bodily cavity.

The present medical device may be intended for short-term, prolonged or long-term contact with living tissue. By “short-term” is meant a duration of less than 24 hours, in accordance with definitions found in ISO 10993-1 for the biological evaluation of medical devices. Furthermore, “prolonged”, according to the same standard, refers to a duration of from 24 hours up to 30 days. Accordingly, by the same standard, by “long-term” is meant a duration of more than 30 days. Thus, in some embodiments the medical device of the invention may be a permanent implant, intended to remain for months, years, or even life-long in the body of a subject.

As used herein the term “implant” includes within its scope any device of which at least a part is intended to be implanted into the body of a vertebrate animal, in particular a mammal, such as a human. Implants may be used to replace anatomy and/or restore any function of the body. Generally, an implant is composed of one or several implant parts. For instance, a dental implant usually comprises a dental fixture coupled to secondary implant parts, such as an abutment and/or a restoration tooth. However, any device, such as a dental fixture, intended for implantation may alone be referred to as an implant even if other parts are to be connected thereto.

By “biocompatible” is meant a material which, upon contact with living tissue, does not as such elicit an adverse biological response (for example inflammation or other immunological reactions) of said tissue.

By “soft tissue” is meant any tissue type, in particular mammalian tissue types, that is not bone or cartilage. Examples of soft tissue for which the medical device is suitable include, but are not limited to, connective tissue, fibrous tissue, epithelial tissue, vascular tissue, muscular tissue, mucosa, gingiva, and skin.

As used herein, the term “compound of a post-transition metal” refers to a chemical entity comprising at least one post-transition metal and at least one additional element. Non-limiting examples of such compounds include oxides comprising post-transition metal, nitrides comprising post-transition metal, alloys of at least one post-transition metal, and salts comprising post-transition metal. The compound may comprise two or more post-transition metals. The term “compound(s) of a post-transition metal” is intended to also refer to compounds incorporating one or more other metals, in particular biocompatible metals such as titanium, in addition to the one or more post-transition metal(s). Hence, as used herein, the term “gallium compound” refers to a chemical entity comprising gallium and at least one additional element. Non-limiting examples of gallium compounds include gallium oxide, gallium nitride, and gallium salts. The gallium and the at least one additional element may be joined by covalent bonding or ionic bonding. The term “gallium compound” is intended to also include compounds incorporating other metals in addition to gallium, in particular titanium. Thus, gallium-titanium oxides, gallium-titanium nitrides etc are included within the definition “gallium compound”.

By analogy, the term “bismuth compound” refers to a chemical entity comprising bismuth and at least one additional element. Non-limiting examples of bismuth compounds include bismuth oxide, bismuth nitride, and bismuth salts. The bismuth and the at least one additional element may be joined by covalent bonding or ionic bonding. The term “bismuth compound” is intended to also include compounds incorporating other metals in addition to bismuth, in particular titanium. Thus, bismuth-titanium oxides, bismuth-titanium nitrides etc are included within the definition “bismuth compound”.

As used herein, “metallic compound” refers to a compound which comprises at least one metal and which has one or more properties which are associated with metals, such as metallic lustrous color. A metallic compound may be formed of a metal and a non-metal, or of two or more metals. Hence, a metallic compound need not comprise non-metallic elements, but may be formed of metallic elements only. In the context of the present invention, at least the following compounds are considered metallic compounds: gallium nitride, gallium-titanium nitride, gallium-titanium, gallium-bismuth-titanium, gallium-bismuth-titanium nitride, bismuth nitride, bismuth-titanium nitride, and bismuth-titanium.

As used herein, “homogeneous layer” refers to a layer having a chemical composition that is uniform in all directions (three dimensions).

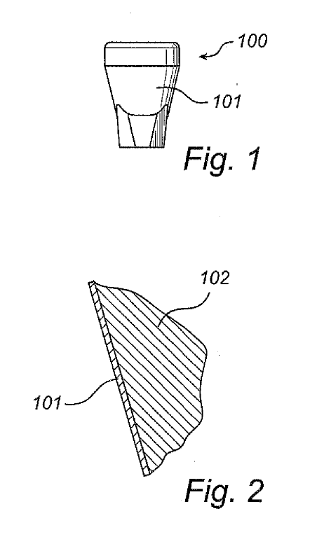

FIGS. 1 and 2 illustrate an embodiment according to the present invention in which the medical device is a dental abutment. The dental abutment 100 comprises a body of substrate material 102 coated with a layer 101 comprising a gallium compound. The layer 101 forms the surface of the abutment intended to face and contact the gingival tissue after implantation.

The medical device of the invention may be made of any suitable biocompatible material, e.g. materials used for implantable devices. Typically the medical device comprises a substrate having a surface which comprises a compound of a post-transition metal, such as a gallium compound or a bismuth compound. The substrate may for example be made of a biocompatible metal or metal alloy, including one or more materials selected from the group consisting of titanium, zirconium, hafnium, vanadium, niobium, tantalum, cobalt and iridium, and alloys thereof. Alternatively, the substrate of the medical device may be made of a biocompatible ceramic, such as zirconia, titania, shape memory metal ceramics and combinations thereof. In embodiments where the medical device is used as or forms part of a dental abutment, the substrate may be made of a metallic material.

In contact with oxygen, the metals titanium, zirconium, hafnium, tantalum, niobium and their alloys instantaneously react to form an inert oxide. Thus, the surfaces of articles of these materials are virtually always covered with a thin oxide layer. The native oxide layer of a titanium substrate mainly consists of titanium(IV) dioxide (TiO₂) with minor amounts of Ti₂O₃, TiO and Ti₃O₄.

Thus, in embodiments where the medical device comprises one or more of titanium, zirconium, hafnium, tantalum, niobium or an alloy of any one thereof, the medical device typically has a native metal oxide surface layer. Such a native metal oxide layer may, in turn, be covered by a thin film comprising the gallium compound. Alternatively, a native metal oxide layer may be modified or replaced with a modified surface layer incorporating a post-transition metal, such as gallium or bismuth, for example a titanium-gallium oxide or titanium-bismuth oxide layer.

In other embodiments of the present invention, the medical device, in particular the substrate, may be made of a biocompatible polymer, typically selected from the group consisting of polyether ether ketone (PEEK), poly methyl methacrylate (PMMA), poly lactic acid (PLLA) and polyglycolic acid (PGA) and any combinations and copolymers thereof.

In embodiments of the invention, the medical device is intended for short-term, prolonged or long-term contact with living tissue. For example, the medical device of the invention may be an implant, typically intended to temporarily or permanently replace or restore a function or structure of the body.

Typically, at least part of the surface of the medical device is intended for contact with soft tissue, and at least part of this soft tissue contact surface has a layer comprising a compound of a post-transition metal, e.g. a gallium compound or a bismuth compound. For example, the medical device may be an implant intended for contact primarily or exclusively with soft tissue, for example a dental abutment. Alternatively, the medical device may be an implant to be inserted partially in bone and partially in soft tissue. Examples of such implants include one-piece dental implants and bone-anchored hearing devices (also referred to as bone anchored hearing aids). Where only part of the implant is intended for contact with soft tissue, it is preferred that the layer comprising the compound of a post-transition metal, e.g. gallium or bismuth compound, is provided at least on a part of a soft tissue contact surface.

The medical device may also be suitable for contact with cartilage.

In other embodiments, the medical device may be intended for contact with bone tissue, e.g. the jawbone, the femur or the skull of a mammal, in particular a human. Examples of such medical devices include dental fixtures and orthopedic implants.

In embodiments of the invention, the compound of a post-transition metal may be a gallium compound and/or a bismuth compound. Suitable compounds include in particular compounds that can be applied on a surface using a thin film deposition technique.

Examples of suitable gallium compounds include gallium oxide, gallium nitride, gallium carbide, gallium selenide, gallium sulphide, and other gallium salts that can be deposited using a thin film deposition, for example gallium chloride, gallium fluoride, gallium iodide, gallium oxalate, gallium phosphate, gallium maltolate, gallium acetate and gallium lactate.

In embodiments of the invention, the gallium compound comprises at least gallium oxide (Ga₂O₃). Gallium oxide may be present in amorphous or crystalline form. Crystalline forms of gallium oxide include α-Ga₂O₃, β-Ga₂O₃, γ-Ga₂O₃, δ-Ga₂O₃, and ε-Ga₂O₃.

Examples of suitable bismuth compounds include bismuth oxide, bismuth nitride, bismuth carbide, bismuth selenide, bismuth sulphide, and other bismuth salts that can be deposited using a thin film deposition, for example bismuth chloride, bismuth fluoride, bismuth iodide, bismuth oxalate, bismuth phosphate, bismuth maltolate, bismuth acetate and bismuth lactate.

The compound of a post-transition metal may optionally include at least one further metal, such as titanium. Hence, in embodiments of the invention, a gallium compound may be selected from the group consisting of gallium oxide, gallium-titanium oxide, gallium nitride, gallium-titanium nitride, gallium-titanium, gallium-bismuth-titanium, and gallium-bismuth-titanium nitride. Similarly, a bismuth compound may be selected from the group consisting of bismuth oxide, bismuth-titanium oxide, bismuth nitride, bismuth-titanium nitride, bismuth-titanium, bismuth-gallium-titanium, bismuth-gallium-titanium-Oxide, and bismuth-gallium-titanium nitride.

Not wishing to be bound by any particular theory, it is believed that upon contact with living tissue and/or body fluids, a layer of for example gallium oxide or gallium nitride (optionally comprising an additional metal such as titanium) exhibits slow, sustained release of gallium ions. Such release may be slower and more sustained compared to the release of gallium ions from a precipitated gallium salt, and may thus provide a more long-term effect with respect to biofilm formation. In addition, a surface layer deposited using a thin film deposition method as used in embodiments of the invention firmly adheres to the underlying substrate and thus avoids problem related to peeling and flaking of the surface layer. Peeling and flaking may give rise to adverse inflammatory response of the surrounding tissue, and in addition may undermine the biofilm prevention effect of the surface layer.

Depending on the intended use of the medical device, different release properties may be desirable. For example, a higher release rate of antimicrobial post-transition metal may be more favorable for short term use, i.e. for a medical device intended for short-term contact with living tissue, compared to a device intended for prolonged or long-term contact. The release rate may be affected by various factors, for example the crystallinity of the compound.

Optionally, in embodiments of the invention the medical device may additionally comprise a salt of a post-transition metal, e.g. a gallium salt selected from the group consisting of gallium acetate, gallium carbonate, gallium chloride, gallium citrate, gallium fluoride, gallium formate, gallium iodide, gallium lactate, gallium maltolate, gallium nitrate, gallium oxalate, gallium phosphate, and gallium sulphate. Alternatively the salt may be a bismuth salt of formed of any of these counterions. Such a salt may be provided as a deposit, e.g. precipitated, on the layer comprising the compound of a post-transition metal.

As mentioned above, the compound of a post-transition metal, e.g. gallium compound or bismuth compound, is typically contained in an applied surface layer. In embodiments of the invention, the compound, optionally including a further metal such as titanium, may constitute the major part of said layer. The atomic concentration (at %) of the elements together forming a compound of post-transition metal may constitute at least 50 at % of the layer, for example at least 70 at %, such as at least 80 at % of the elements of the layer.

The atomic concentration of post-transition metal, e.g. gallium, in the layer may be in the range of from 5 at % up to 50 at %, for example at least 10 at %, at least 15 at %, at least 20 at %, at least 35 at % or at least 30 at %, and up to for example 50 at %, such as up to 45 at %, or up to 40 at %. However in embodiments of the invention, the atomic content of the post-transition metal may be less than 5 at %. For example, the atomic content of the post-transition metal may be in the range of from 0.01 to 20 at %, e.g. from 0.05 to 15 at %, such as from 0.1 to 15 at %. For example, in embodiments where the compound of post-transition metal comprises gallium, the content of gallium in the layer may be at least 0.05 at % or at least 0.1 at %, for example at least 0.3 at % or at least 0.5 at %. In embodiments where the compound of post-transition metal comprises bismuth, the content of bismuth in the layer may be for instance at least 0.1 at %, at least 0.2 at %, at least 1 at % or at least 1.5 at %.

When the gallium compound is substantially pure gallium oxide (Ga₂O₃), the maximum content of gallium in the layer is 40 at %, and the maximum content of oxygen in the layer is 60 at %. However, impurities and contamination, for example carbon, may be present at up to 20 at %. If the gallium compound is gallium-titanium oxide, part of the gallium is replaced with titanium and so the total content of gallium and titanium would be 40 at %.

Where the gallium compound is substantially pure gallium nitride (GaN), the maximum content of gallium in the layer is 50 at %, and the maximum content of nitrogen is 50 at %. Contaminations may be present as described above. If part of the gallium is replaced with another metal, such as titanium, the total content of gallium and said other metal may still be 50 at %, or it may be higher, e.g. up to 75%, if the nitrogen content is low. As an example, the layer may contain 50 at % nitrogen, 25 at % titanium and 25 at % gallium.

The atomic concentration may be measured for example to a depth of 40 nm or less, and preferably not more than the layer thickness. The atomic concentration can be measured using X-ray photoelectron spectroscopy (XPS).

As mentioned above, upon contact with living tissue, some post-transition metal, such as gallium or bismuth may be released from the surface of the medical device over time. Hence after implantation the content of post-transition metal and possibly also of other materials present on the surface of the medical device may change over time.

In some embodiments, the layer may in addition to the compound of a post-transition metal include one or more other elements or compounds (a dopant), for example at a content of 10 at % or less, as will be described in more detail below. The layer comprising the gallium compound or other compound of a post-transition metal may also contain impurities or contamination, for example carbon, typically in an amount of 20 at % or less, e.g. 15 at % or less, or 10 at % or less. Such contamination may originate from the packaging. It may be noted that wet packaging, in which the surface may be protected by water, ethanol or the like, reduces the amount of contamination by carbon, compared to dry packaging where the surface is exposed to air which normally contains volatile hydrocarbons. Contamination may also be present on the surface of the substrate before the layer comprising the gallium compound is applied. The level of contamination, typically represented by the atomic concentration of carbon, may be reduced by cleaning the surface before applying the compound of post-transition metal, and optionally after applying the compound of post-transition metal and/or by avoiding further contaminating the surface before measuring the atomic concentration of elements on the surface.

Table 1 summarizes possible atomic concentration ranges for a layer comprising bismuth nitride, bismuth-titanium nitride, gallium oxide, gallium-titanium oxide, gallium nitride or gallium-titanium nitride, respectively.

TABLE 1
Exemplary atomic concentrations of various elements
of a gallium compound.

| | Atomic concentration (at %) | | |
|---|---|---|---|
| | | gallium oxide/ | Gallium nitride/ |
| | Bismuth nitride/ | gallium-titanium | Gallium-titanium |
| | Bismuth-titanium nitride | oxide | nitride |
| Ga | — | 5-40 at % | 0.1-50 at %; |
| | | | e.g. 5-50 at % |
| Bi | 0.1-50 at % | — | — |
| Ti | 0-75 at %; | 0-35 at % | 0-75 at %; |
| | e.g. 25-75 at % | | e.g. 0-45 at %; e.g. |
| | | | 10-60 at % |
| O | — | 7.5-60 at % | — |
| N | 5-50 at %; | — | 5-50 at %; |
| | e.g. 20-50 at % | | e.g. 20-50 at % |

In embodiments of the invention, the compound of a post-transition metal may primarily comprise a post-transition metal and an additional metal, such as titanium. For example, the surface layer may comprise titanium-gallium or titanium-bismuth as the compound of a post-transition metal. In such embodiments, the content of post-transition metal may be in the range of from 0.01 to 20 at %, e.g. from 0.05 to 15 at %, such as from 0.1 to 15 at %, as described above. Further, the titanium content may be in the range of from 10 to 99.9 at %, e.g. 10 to 60 at %, or 10 to 30 at %. The layer may additionally contain nitrogen at a content of from 0 to 15 at %. For example, the nitrogen content of the layer may typically be in the range of from 0 to 5 at %. Furthermore, depending on the handling (e.g. cleaning steps) and/or analytical technique used for determining the surface chemical composition, the layer may additionally have a relatively high but superficial oxygen content, e.g. from 0 up to about 60 at %.

In some embodiments, the surface layer consists essentially of one or more of the compound(s) of a post-transition metal mentioned above. In accordance with the above, “consists essentially of” here means that the layer contains lithe or no other material (dopants, contaminants, etc) except the one or more compound(s) of a post-transition metal, only for example up to 10 at %, e.g. up to 5 at %, such as up to 2 at % or up to 1 at % of other material.

In general, the layer comprising the compound of a post-transition metal, e.g. a gallium compound or bismuth compound, is free of carrier material such as polymers, solvents, etc.

The layer comprising the compound of a post-transition metal such as a gallium compound or a bismuth compound may have a thickness in the range of from 1 nm to 1.5 μm, for example 0.1 to 1 μm, in particular from 0.3 to 1 μm. A layer having a thickness of at least 1 nm may provide sufficient antimicrobial effect. Increasing layer thickness may provide a whiter color, which may be desirable for dental applications. However, also a layer having a thickness of from about 10 nm may be more aesthetically advantageous than present commercial dental abutments. For example, a gallium oxide layer of 40 nm has a deep bronze color which would be less visible through a patient's gingiva than current grey-metallic titanium abutments.

Where mainly an antimicrobial effect is sought, the layer containing the compound of a post-transition metal may optionally have a thickness of from 10 to 100 nm, or optionally up to 300 nm. On the other hand, where the aesthetic appearance of e.g. a dental abutment is of high importance, a layer thickness in the range of from 0.5 to 1.5 μm, e.g. from 0.7 to 1.5 μm or from 0.7 to 1 μm may be particularly contemplated. However also thinner layers may provide an acceptable color appearance and which at least may be more advantageous than prior art dental abutments.

The layer may be a dense layer, i.e. a non-porous layer.

In embodiments of the invention, a salt of a non-toxic post-transition metal, optionally forming a further layer, may be provided on at least a portion of a thin-film deposited layer comprising the above-described compound of non-toxic post-transition metal. For example, a solution of at least one gallium salt may be applied onto a thin-film deposited layer of the gallium compound, and allowed to evaporate. Such embodiments may provide a high initial release of gallium upon contact with living tissue, which may be advantageous in many instances, for short-term, prolonged as well as for long-term tissue contact.

In embodiments of the invention, a surface layer of the medical device may contain at least one additional element(s) or compound(s), for example a bioactive element or compound that may further enhance tissue healing or function. Such elements or compounds may be included as a dopant in the layer comprising the compound of a post-transition metal, typically at a content of 10 at % or less. Alternatively such element(s) or compound(s) may be applied as a separate layer, e.g. on the layer comprising the compound of a post-transition metal.

In embodiments of the invention, the substrate may have a rough surface on which a layer comprising the compound of a post-transition metal is arranged. Since the layer comprising the compound of a post-transition metal may be thin, e.g. 100 nm or less, it may have good conformal step coverage, meaning that the layer follows the underlying surface roughness and substantially preserves it, without making it smoother. However, in embodiments where the layer comprising the compound of a post-transition metal is relatively thick, it may reduce the roughness of the underlying substrate surface.

The substrate surface roughness, and hence optionally also the surface of the medical device formed by the layer comprising the compound of a post-transition metal, may have an average surface roughness Rₐ of at least 0.05 μm, typically at least 0.1 μm, for example at least 0.2 μm. Since surfaces having an average surface roughness (Rₐ) of at least 0.2 μm are believed to be more susceptible of biofilm formation, a layer comprising a compound of a post-transition metal, e.g. a gallium compound or a bismuth compounds as described herein may be particularly advantageous for medical devices having a surface roughness of at least 0.2 μm, and may be increasingly useful for preventing biofilm formation on medical devices having even higher surface roughness. As an example, a dental abutment comprising a titanium substrate may have a surface roughness of about 0.2-0.3 μm. A surface layer of e.g. a gallium compound having a thickness of about 40 nm may substantially preserve this surface roughness (which may be desirable e.g. in order to facilitate a firm anchorage of the implant in the surrounding tissue) but may prevent biofilm formation on the implant surface and hence reduce the risk for infection and periimplantitis.

The layer comprising a compound of a post-transition metal may be formed by applying the compound of a post-transition metal onto the surface of a medical device, to form a surface layer. The compound of a post-transition metal may be applied using known deposition techniques, especially thin film deposition techniques. Suitable techniques may include physical deposition, chemical deposition and physical-chemical deposition. One example of such techniques is atomic layer deposition (ALD) which can be used to provide e.g. a gallium oxide layer on a substrate surface (Nieminen et al, 1996; Shan et al, 2005). Other techniques that may be used for depositionare chemical vapor deposition (Pecharroman et al, 2003; Kim et al, 2010), spray pyrolysis deposition (Kim & Kim, 1987; Hao et al, 2004), arc discharge deposition (Heikman et al, 2006), electron beam deposition (Al-Kuhaili et al, 2003), pulsed laser deposition (Orita et al, 2002), thermal evaporation deposition (Karaagac & Parlak, 2011), sputter deposition (Kim & Holloway, 2004) and molecular beam deposition (Passlack et al, 1995). Physical deposition, chemical deposition, and physical-chemical deposition techniques may also be used for incorporating additional element(s) into a layer of compound(s) of post-transition metal, e.g. as dopants, in order to enhance healing or regeneration of the tissue contacting the medical device.

ALD and other thin film deposition techniques are associated with several advantages for the deposition of the compound(s) of a post-transition metal, such as controlled layer thickness, controlled composition, high purity, conformal step coverage, good uniformity (resulting in a homogeneous layer), and good adhesion. In particular, PVD may provide dense layers with excellent adhesion to the underlying substrate.

### EXAMPLES

### Example 1A

### Production of Gallium Oxide Coated Specimens

Coins of commercially pure (cp) titanium (grade 4) were manufactured and cleaned before deposition of a 40 nm thick layer of amorphous Ga₂O₃ using atomic layer deposition (Picosun, Finland) with precursors of GaCl₃ and H₂O, respectively. Specimens were thereafter packaged in plastic containers, and sterilized with electron beam irradiation.

### Example 1B

### Surface Characterization of Gallium Oxide Coated Specimens

For all surface characterization experiments, eight specimens each of commercially pure (cp) titanium, Ga₂O₃ coated cp titanium produced as described above, and commercially available TIN coated cp titanium, were prepared as described in Example 1 (cleaned, coating using ALD in the case of the Ga₂O₃ coated specimens, packaged, and sterilized). The TiN coated specimens were included for comparison since it is known that a TiN coating provides a weakly antibacterial effect.

It was found that the surface morphology and surface roughness was unaltered by the ALD coating, but there was a slight increase of hydrophobic properties.

a) Surface Chemistry

Surface morphology and surface chemistry was analyzed with environmental scanning electron microscopy (XL30 ESEM, Philips, Netherlands)/energy dispersive spectroscopy (Genesis System, EDAX Inc., USA) at an acceleration voltage at 10-30 kV. Elements detected on the surface of the Ga₂O₃ coated specimens were oxygen (O), gallium (Ga), and titanium (Ti). Ga concentrations varied between 4 to 9 atomic % (at %), as measured with acceleration voltages at 30 kV and 10 kV, respectively. The analytical depth with this technique is estimated to be approximately 1 μm, i.e. much deeper than the layer thickness. No differences in terms of surface morphology could be detected between commercially pure titanium controls and the Ga₂O₃ coated titanium.

Additionally, surface chemistry was analyzed with X-ray photoelectron spectroscopy (XPS, Physical Electronics, USA), which is a more surface sensitive technique than energy dispersive spectroscopy. As X-ray source monochromatic AlKα was used. The beam was focused to 100 μm. Elements detected were oxygen (O), gallium (Ga), and carbon (C). Gallium concentrations varied between 34 and 37 at %. Oxygen concentrations varied between 47 and 50 at %. The analytical depth with this technique is estimated to be approximately 5-10 nm. The results are summarized in Table 2.

TABLE 2
Atomic concentration of detected elements using XPS

| Element | At % detected using XPS (depth 5-10 nm) |
|---|---|
| Ga | 34-37 at % |
| O | 47-50 at % |
| C | 13-18 at % |

b) Surface Morphology

Surface roughness was measured with surface profilometry (Hommel T1000 wave, Hommelwerke GmbH, Germany). A vertical measuring range of 320 μm, and an assessment length of 4.8 mm were used. Two specimens of each type were included in the analysis, and three measurements per specimen were performed. The surface roughness Rₐ was calculated after using a filtering process, with cut-off at 0.800 mm. The results are presented in Table 3.

TABLE 3
Surface roughness (Rₐ) ± standard deviations (SD)

| Test specimens | Rₐ (μm) |
|---|---|
| TiN coated titanium | 0.28 ± 0.01 |
| Ga₂O₃ coated titanium | 0.31 ± 0.04 |
| Uncoated titanium | 0.34 ± 0.03 |

c) Wettability

In order to investigate the wettability, the contact angle was measured using a contact angle measuring system (Drop Shape Analysis System DSA 100, Kruss GmbH, Germany). Measurements were performed with deionized water. The results indicate that all specimens were hydrophobic (>90°), see Table 4.

TABLE 4
Contact angles (°) ± standard deviations (SD)

| Test specimens | Contact angle (°) |
|---|---|
| TiN coated titanium | 95.0 ± 1.4 |
| Ga₂O₃ coated titanium | 97.7 ± 2.2 |
| Uncoated titanium | 92.0 ± 2.8 |

### Example 1C

### Antimicrobial Effect of Gallium Oxide-Coated Surfaces

It was found that a titanium body having a surface comprising gallium (Ga) in the form gallium oxide can prevent the growth of Pseudomonas aeruginosa and Staphylococcus aureus on and around a surface and thus may be useful in preventing detrimental infection around e.g. a dental abutment implanted into the gingiva.

a) Inhibition of Bacterial Growth on Streak Plate

In a first experiment commercially pure titanium coins (φ 6.25 mm) with or without a gallium oxide coating were placed on agar plates containing homogeneously distributed colonies of Pseudomonas aeruginosa. After incubation for 24 hours at 37° C. there was a 4 mm wide visible colony free zone surrounding the gallium oxide coins, in contrast to the titanium coins that were surrounded by bacterial colonies.

b) Inhibition of Bacterial Growth Using Film Contact Method

In a second experiment, a film contact method (Yasuyuki et al, 2010) was used. Streak plates of Pseudomonas aeruginosa (PA01) or methillicin resistant Staphylococcus aureus (MRSA) were made and 1 colony was inoculated to 5 ml tryptic soy broth (TSB) in culture tubes and grown under shaking conditions for 18 hours. Cell density was measured in a spectrophotometer at OD 600 nm and counted using a cell counting chamber. The cell culture was adjusted with sterile TSB to 1-5×10⁶ cells/ml. Specimens of commercially pure (cp) titanium coins (φ 6.25 mm), cp titanium coins with a gallium oxide coating, or cp titanium coins with a commercially available titanium nitride (TiN) coating were aseptically prepared and put in respective well of a 12 well plate. Thin transparent plastic film was punched, and sterilized using 70% ethanol and UV irradiation on each side. A 15 μl drop of bacteria in TSB was applied on each specimen. One thin plastic film per specimen was placed over the bacteria on the specimens so that the bacterial solution was evenly spread over the specimen surface, ensuring good contact. After incubation for 24 hours at 30±1° C., the film of each specimen was aseptically removed and washed by pipetting 1 ml PBS over the surface into a separate 2 ml eppendorf tube per specimen. The specimens were transferred to the same eppendorf tubes as used when washing the film. First each specimen surface was washed by pipetting the very same PBS as the film was previously washed with. Next, the specimens were sonicated and for 1 minute and vigorously vortexed for 1 minute in the very same tube as previously used when washing the film. Serial dilutions and plate count were performed. Plates were incubated for 24 hours and colony numbers counted and recorded. The antibacterial activity of gallium oxide coated titanium was determined to 92% reduction against PA01 and 71% reduction against MRSA, compared to titanium, see Tables 5 and 6.

TABLE 5
Viable counts (cfu/ml) ± standard deviations (SD) after 24 hours
incubation of test specimens against Pseudomonas Aeruginosa (PA01).

| Test specimens | PA01 (cfu/ml ± SD) 24 h |
|---|---|
| TiN coated titanium | 1.6E+08 ± 1.2E+08 |
| Ga₂O₃ coated titanium | 7.4E+07 ± 2.8E+06 |
| Uncoated titanium | 1.0E+09 ± 6.0E+08 |

TABLE 6
Viable counts (cfu/ml) ± standard deviations (SD) after 24 hours
against methillicin resistant Staphylococcus aureus (MRSA).

| Test specimens | MRSA (cfu/ml ± SD) 24 h |
|---|---|
| TiN coated titanium | 5.0E+08 ± 6.0E+07 |
| Ga₂O₃ coated titanium | 2.2E+08 ± 3.7E+07 |
| Uncoated titanium | 7.6E+08 ± 6.8E+07 |

c) In Situ Effect on a Biofilm

In a third experiment, the antibacterial activity of titanium discs, with or without a gallium oxide coating, was evaluated in situ using Live/Dead® BacLight™ stain (Life Technologies Ltd, UK). Streak plates of Pseudomonas aeruginosa (PA01) were made and 1 colony was inoculated to 5 ml TSB in culture tubes and grown under shaking conditions for 18 hours. Cell density was measured in a spectrophotometer at OD 600 nm and adjusted with sterile TSB to 1×10⁶ cells/ml. 400 μl bacteria were aliquoted into 8-chambered slides. The biofilm was allowed to be formed during 24 hours at 35±2° C. Specimens of commercially pure (cp) titanium coins (φ 6.25 mm), cp titanium coins with a gallium oxide coating, or cp titanium coins with a titanium nitride (TiN) coating were aseptically prepared and applied onto the biofilm. The antibacterial activity was analyzed in situ using the Live/Dead® stain.

In situ analyses indicated that both titanium nitride and gallium oxide coatings have an anti-biofilm activity compared with uncoated titanium in terms of viability. At 24 hour analysis, it was visualized that the typical mushroom structure of biofilms had disappeared for titanium nitride and gallium oxide. It was also found that more dead cells were seen on gallium oxide than on titanium nitride.

### Example 2A

### Production of Nitride or Metal Coated Specimens

Coins of commercially pure (cp) titanium (grade 4) were manufactured and cleaned before deposition of a 0.5-1 μm thick layer of either titanium bismuth (TiBi), titanium nitride with bismuth (TiNBi), titanium gallium (TiGa) or titanium nitride with gallium (TiNGa) using physical vapor deposition (PVD). Targets of TiBi and TiGa were used for TiBi/TiNBi and TiGa/TiNGa, respectively. The specimens were thereafter packaged in plastic containers, and sterilized with electron beam irradiation.

### Example 2B

### Surface Characterization of Nitride or Metal Coated Specimens

For these surface characterization experiments, specimens of commercially pure (cp) titanium, TiBi coated titanium, TiNBi coated titanium, TiGa coated titanium and TiNGa coated titanium produced as described in Example 2A were used.

It was found that the surface morphology and surface roughness was unaltered by the PVD coating, and that both uncoated titanium and the coated specimens had hydrophilic properties.

The nitride specimens (TiNGa, TiNBi) had a goldish color, similar to the color of TiN surfaces on commercially available dental implants. It is expected that a BiN or a GaN surface layer would have a goldish appearance as well. In contrast, the TiBi and TiGa specimens had a grey-metallic color, similar to that of the uncoated titanium specimens.

a) Surface Morphology and Surface Chemistry

Surface morphology and surface chemistry were analyzed with environmental scanning electron microscopy (XL30 ESEM, Philips, Netherlands)/energy dispersive spectroscopy (Genesis System, EDAX Inc., USA) at an acceleration voltage at 10 kV. Concentrations of bismuth (Bi) and gallium (Ga) on the coated specimens (TiBi, TiNBi, TiGa, TiNGa) were found to be up to 12.6 at % for Bi and 1.1 at % for Ga, as measured with acceleration voltages at 10 kV. Other elements detected on the surfaces were titanium (Ti), nitrogen (N), oxygen (O), and carbon (C). The analytical depth with this technique is estimated to be approximately 1 μm, i.e. possibly deeper than the layer thickness. No differences in terms of surface morphology could be detected between commercially pure titanium controls and the coated titanium (TiBi, TiNBi, TiGa, TiNGa).

Additionally, surface chemistry was analyzed with X-ray photoelectron spectroscopy (XPS, Physical Electronics, USA), which is a more surface sensitive technique than energy dispersive spectroscopy. As X-ray source monochromatic AlKα was used. The beam was focused to 100 μm. Elements detected were titanium (Ti), nitrogen (N), carbon (C), and oxygen (O), as well as bismuth (Bi) (for TiBi and TiNBi specimens) and gallium (Ga) (for TiGa and TiNGa specimens). Bi and Ga concentrations were detected to be up to 3.3 at % and 0.4 at %, respectively. The analytical depth with this technique is estimated to be approximately 5-10 nm. The results are summarized in Table 7.

TABLE 7
Summary of surface chemistry analyses of titanium controls and coated surfaces (TiBi,
TiNBi, TiGa, TiNGa). Values (at %) describe the ranges of elementary concentrations
(n = 6).

| Surface | | | | | | |
|---|---|---|---|---|---|---|
| analytical | Elements | Surface coating | | | | |
| technique | detected | Ti (uncoated) | TiBi | TiNBi | TiGa | TiNGa |
| EDX | Ti | 83.4-84.6 | 72.4-93.1 | 58.4-73.2 | 98.9-99.5 | 58.9-59.2 |
| | N* | 0-11.8 | 0-10.1 | 26.1-41.3 | 0-12.3 | 40.9-41.1 |
| | Bi | — | 5.9-12.6 | 0.2-0.7 | — | — |
| | Ga | — | — | — | 0.5-1.1 | — |
| XPS | Ti | 21.0-25.9 | 16.1-24.1 | 27.8-28.5 | 15.4-24.6 | 18.2-28.9 |
| | N | 0.7-2.0 | 0-1.8 | 26.0-27.0 | 1.9-2.7 | 24.6-28.6 |
| | Bi | — | 1.9-3.3 | 0.2 | — | — |
| | Ga | — | — | — | 0.3-0.4 | 0.1-0.3 |
| | C | 18.8-27.7 | 15.8-33.1 | 16.0-17.8 | 18.6-45.9 | 18.2-28.9 |
| | O | 49.2-55.5 | 46.5-56.8 | 27.7-29.2 | 36.2-54.8 | 21.5-24.2 |

*The titanium signal (TiL) and the nitrogen signal (NK) are closely located, which contributes to the variance in nitrogen (N) concentration.

b) Surface Roughness

The surface roughness of specimens produced according to Example 2A was measured with surface profilometry (Hommel T1000 wave, Hommelwerke GmbH, Germany). A vertical measuring range of 320 μm and an assessment length of 4.8 mm were used. Three specimens of each type were included in the analysis, and three measurements per specimen were performed. The surface roughness in terms of arithmetic mean value of vertical deviations of the roughness profile from a mean line (Rₐ) was calculated after using a filtering process, with cut-off at 0.800 mm. The results are presented in Table 8.

TABLE 8
Surface roughness (Ra) ± standard deviations (SD), n = 9.

| Test specimen | Ra (μm) |
|---|---|
| Uncoated titanium | 0.23 (0.04) |
| TiBi coated titanium | 0.24 (0.05) |
| TiNBi coated titanium | 0.22 (0.03) |
| TiGa coated titanium | 0.23 (0.05) |
| TiNGa coated titanium | 0.23 (0.02) |

c) Wettability

In order to investigate the wettability of specimens produced according to Example 2A, the contact angle was measured using a contact angle measuring system (Drop Shape Analysis System DSA 100, Kruss GmbH, Germany). Measurements were performed with deionized water. The result showed that all specimens were hydrophilic (<90°) although variations between test specimens were observed, see Table 9.

TABLE 9
Contact angles (°) ± standard deviations (SD)

| Test specimens | Contact angle (°) |
|---|---|
| Uncoated titanium | 63 (5) |
| TiBi coated titanium | 50 (6) |
| TiNBi coated titanium | 66 (5) |
| TiGa coated titanium | 41 (1) |
| TiNGa coated titanium | 59 (2) |

### Example 2C

### Release of Gallium and Bismuth from Surface Coatings

Release of gallium (Ga) or bismuth (Bi) from all surface coatings (TiBi, TiNBi, TiGa, TiNGa) was confirmed in experiments. At a pH of 7.4 low amounts of Ga and Bi, respectively, were released, but at pH 5.0 the release of Bi increased significantly.

For all release experiments, specimens of commercially pure (cp) titanium, TiBi coated titanium, TiNBi coated titanium, TiGa coated titanium and TiNGa coated titanium produced as described in Example 2A were used. The release experiments were performed by immersing specimens in either phosphate buffer (pH 7.4) or acetate buffer (pH 5.0). The pH of 7.4 was intended to simulate a physiologic neutral condition (pH 7.4). The acidic pH (5.0) was intended to simulate a condition that may occur locally in the tissue the case of inflammation and/or infection. Glass bottles containing 10 ml buffer were used with one specimen per bottle. The bottles were placed on a shanking table at 37° C. during the experiment. After 1, 4, 7, and 24 days liquid samples were taken and the Bi and Ga concentrations were analyzed using Inductively Coupled Plasma—Sector Field Mass Spectrometry (ICP-SFMS). The results are presented in Table 10.

TABLE 10
Release of bismuth (Bi) or gallium (Ga) to acetate buffer (pH 5.5) or phosphate buffer
(pH 7.4). Figures are mean values (n = 2), standard deviations within parentheses.

| | Release of Bi (μg/l) | | | | Release of Ga (μg/l) | | | |
|---|---|---|---|---|---|---|---|---|
| Time | TiBi coated Ti | | TiNBi coated Ti | | TiGa coated Ti | | TiNGa coated Ti | |
| (days) | pH 5.0 | pH 7.4 | pH 5.0 | pH 7.4 | pH 5.0 | pH 7.4 | pH 5.0 | pH 7.4 |
| 1 | 2840 | 16.2 | 1150 | 3.6 | 0.9 | 1.3 | 8.4 | 11.9 |
| | (240) | (0.1) | (28) | (0.2) | (0.1) | (0.1) | (1.4) | (1.2) |
| 4 | 3225 | 25.6 | 1660 | 3.9 | 0.7 | 1.3 | 12.7 | 12.4 |
| | (21) | (7.9) | (806) | (1.2) | (0.1) | (0.2) | (0.4) | (1.7) |
| 7 | 2700 | 22.9 | 1470 | 2.6 | 0.7 | 1.6 | 13.3 | 12.4 |
| | (141) | (1.6) | (438) | (0.3) | (0.1) | (0.1) | (2.1) | (0.4) |
| 24 | — | 10.3 | — | 8.3 | — | 2.1 | — | 14.7 |
| | | (0.1) | | (2.0) | | (0.3) | | (0.9) |

### Example 2D

### Antimicrobial Effect of Nitride or Metal Coated Specimens

It was found that a titanium body having a surface comprising gallium (Ga) or bismuth (Bi) in the form TiGa, TiNGa, TiBi or TiNBi can prevent the growth of Pseudomonas aeruginosa and Staphylococcus aureus on a surface and thus may be useful in preventing detrimental infection around e.g. a dental abutment implanted into the gingiva.

a) Antimicrobial Effect of Bismuth (Bi) and Gallium (Ga) Against Different Bacteria Species

Streak plates and characterization by gram-staining were done. MRSA and PA01 were grown on Tryptic Soy agar (TSA) plates and S. sanguinis on horse blood plates for up to 24 hours whereas P. gingivalis was grown between 4 and 5 days on Fastidious Anaerobic agar (FAA) plates. Fivefold dilution in at least five steps of gallium nitrate (Ga(NO₃)₃) or bismuth chloride (BiCl₃) was done. Bismuth salt had to be suspended in DMSO in order to enable salt to dissolve in culture media.

Equal amounts of bacterial solution and antimicrobial salt (in fivefold dilution) were mixed and incubated at 35±2° C.: MRSA, PA01, and S. sanguinis for 24 hours and P. gingivalis for 4 or days. After incubation, the concentration where no visual growth could be detected was documented. Results are summarized in Table 11.

TABLE 11
Minimum Inhibitory Concentration (MIC) values in μg/ml.

| | MRSA | PA01 | S. sanguinis | P. gingivalis |
|---|---|---|---|---|
| Ga(NO₃)₃ | 1000 | 200 | 1000 | 40 |
| (μg/ml) | | | | |
| BiCl₃ (μg/ml) | 20* | 20* | 40 | 1000 |
| | 900* | 900* | | |
| | 40* | 200* | | |

*The MIC values differed at the three different runs, hence MIC values from all runs are included.

The MIC values indicate that both Ga and Bi exert an antibacterial effect against various bacterial species, although the effect varies for different species. Variation in MIC values for BiCl₃ against MRSA and PA01 was probably caused by inadequate dissolution of the BiCl₃ (high values indicating that the salt was not completely dissolved and was thus unavailable for inhibition).

b) Inhibition of Bacterial Growth Using Film Contact Method

In another experiment, a film contact method (Yasuyuki et al, 2010) was used. Streak plates of Pseudomonas aeruginosa (PA01) were made and 1 colony was inoculated to 5 ml tryptic soy broth (TSB) in culture tubes and grown under shaking conditions for 18 hours. Cell density was measured in a spectrophotometer at OD 600 nm and counted using a cell counting chamber. The cell culture was adjusted with sterile TSB to 1-5×10⁶ cells/ml. Specimens of commercially pure (cp) titanium coins (φ 6.25 mm) and cp titanium coins with coatings of TiBi, TiNBi, TiGa or TiNGa were aseptically prepared and put in respective well of a 12 well plate. Thin transparent plastic film was punched, and sterilized using 70% ethanol and UV irradiation on each side. A 15 μl drop of bacteria in TSB was applied on each specimen. One thin plastic film per specimen was placed over the bacteria on the specimens so that the bacterial solution was evenly spread over the specimen surface, ensuring good contact. After incubation for 24 hours at 30±1° C., the film of each specimen was aseptically removed and washed by pipetting 1 ml PBS over the surface into a separate 2 ml eppendorf tube per specimen. The specimens were transferred to the same eppendorf tubes as used when washing the film. First each specimen surface was washed by pipetting the very same PBS as the film was previously washed with. Next, the specimens were sonicated and for 1 minute and vigorously vortexed for 1 minute in the very same tube as previously used when washing the film. Serial dilutions and plate count were performed. Plates were incubated for 24 hours and colony numbers counted and recorded. The antibacterial activity of TiBi, TiNBi, TiGa, or TiNGa coated titanium specimens was determined to >99.5% reduction against PA01 compared to titanium, see Table 12.

TABLE 12
Antibacterial activity was calculated as percent reduction (compared
to uncoated titanium) after 24 hours incubation of test specimens
against Pseudomonas Aeruginosa (PA01).

| Test specimens | Reduction of PA01 (%) |
|---|---|
| TiBi coated titanium | 99.804 |
| TiNBi coated titanium | 99.998 |
| TiGa coated titanium | 99.551 |
| TiNGa coated titanium | 99.524 |

### Example 2E

### Cytotoxicity of Nitride or Metal Coated Specimens

All surface coatings (TiBi, TiNBi, TiGa, TiNGa) were found to enhance fibroblast mitochondrial activity, i.e. cell viability, compared to uncoated titanium surfaces, using a cytotoxicity test.

Cytotoxicity was assessed with a (3-(4,5-dimethylthiazol-2-yl)-5-(3-carboxymethoxyphenyl)-2-(4-sulfophenyl)-2H-tetrazolium (MTS) assay using human fibroblasts (MRC-5). An MTS assay measures the mitochondrial activity in cells and correlates to the viability/the number of cells. For cytotoxicity experiments, specimens of commercially pure (cp) titanium, TiBi coated titanium, TiNBi coated titanium, TiGa coated titanium and TiNGa coated titanium produced as described in Example 2A were used.

The cells were subcultured according to guidelines from the American Type Culture Collection. They had reached approximately 80% confluence when they were used in the experiment. Six coins of each specimen were aseptically placer in a 24 well plate, 1 coin per well. 100 μl of MRC-5 cells at 1×10⁵ cells/ml were added to each disc and controls. The plates were then incubated for one hour to allow cell attachment before 1 ml complete cell culture media was added to each well. The plates were then incubated for 24 hours before analysis. For the MTS assay 4 coins of each specimen were transferred from the 24 well plates to 48 well plates. 500 μl MTS assay reagent was added to each well and were incubated for 4 hours. Absorbance was then read at 490 nm. The absorbance values for each specimen were normalized to the uncoated titanium control in percentage set to 100% (Table 13).

The remaining 2 coins of each specimen were used for DAPI staining and fluorescence microscopy, in order to verify the MTS assay result. DAPI is a fluorescent stain that binds to cell nucleus and can thus be used to visualize cells on metals.

TABLE 13
Mitochondrial activity (i.e. viability) as measured with an MTS
assay for human fibroblasts cultured on uncoated and coated titanium
specimens. Mean values, standard deviations within parentheses.

| | Percent of mitochondrial activity compared to |
|---|---|
| Test specimens | uncoated titanium (uncoated Ti = 100%) |
| TiBi coated titanium | 932 (118) |
| TiNBi coated titanium | 204 (21) |
| TiGa coated titanium | 136 (9) |
| TiNGa coated titanium | 179 (14) |

In conclusion, it has been experimentally demonstrated that a surface layer comprising a compound of a post-transition metal, here a compound of gallium, bismuth, or both, can be applied on a biocompatible metal substrate (titanium) to provide an antimicrobial effect against several bacterial species. It has also been shown that there is a release of post-transition metal from the surface layer at physiologically neutral conditions. Furthermore, at acidic conditions, which may occur very locally in the tissue in the case of inflammation and/or infection, the release of post-transition metal may even be significantly increased. Finally, the tested surface layers are non-toxic to human fibroblast cells and may in fact enhance fibroblast mitochondrial activity.

The person skilled in the art realizes that the present invention by no means is limited to the preferred embodiments described above. On the contrary, many modifications and variations are possible within the scope of the appended claims.

Additionally, variations to the disclosed embodiments can be understood and effected by the skilled person in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measured cannot be used to advantage.

### REFERENCES

1. M. F. Al-Kuhaili, S. M. A. Durrani, E. E. Khawaja. Appl Phys Lett 83 (2003) 4533-4535.
2. J. Hao, Z. Lou, I. Renaud, M. Cocivera. Thin Sold films 467 (2004) 182-185.
3. S. Heikman, S. Keller, U. K. Mishra. J Crystal Growth 293 (2006) 335-343.
4. Y. Kaneko, M. Thoendel, O. Olakanmi, B. E. Britigan and P. K. Singh, The Journal of Clinical Investigation, Vol 117 (2007) 877-888.
5. H. Karaagac, M. Parlak. J Mater Sci Mater Electron 22 (2011) 1426-1432.
6. HG. Kim, W. T. Kim. J Appl Physics 62 (1987) 2000-2002.
7. J. H. Kim, P. H. Holloway. J Appl Physics 95 (2004) 4787-4790.
8. Y. H. Kim, H. Ruh, Y. K. Noh, M. D. Kim, J. E. Oh. J Appl Physics 107 (2010) 063501-1-4.
9. M. Nieminen, L. Niinisto and E. Rauhala. J Mater Chem 6 (1996) 27-31.
10. O. Olakanmi J. S. Gunn, S. Su, S. Soni, D. J. Hassett, B. E. Britigan. Antimicrobial agents and Chemotherapy 54 (2010) 244-253.
11. M. Orita, H. Hiramatsu, H. Ohta, M. Hirano, H. Hosono. Thin solid films 411 (2002) 134-139.
12. M. Passlack, M. Hong, E. F. Schubert, J. R. Kwo, J. P. Mannaerts et al. Appl Phys Lett 66 (1995) 625-627.
13. C. Pecharroman, F. Gracia, J. P. Holgado, M. Ocana, A. R. Gonzalez-Elipe et al. J Appl Physics 93 (2003) 4634-4645.
14. F. K. Shan, G. X. Liu, W. J. Lee, G. H. Lee, I. S. Kim et al. J Appl Physics 98 (2005) 023504-1-6.
15. M. Yasuyuki, K. Kunihiro, S. Kurissery, N. Kanavillil, Y. Sato, Y. Kikuchi. Biofouling 26 (2010) 851-858.

1. A medical device intended for contact with living tissue, comprising a substrate having a surface layer comprising one or more compound(s) of a non-toxic post-transition metal.

2. The medical device according to claim 1, wherein said living tissue is soft tissue.

3. The medical device according to claim 1, wherein said compound of a non-toxic post-transition metal also comprises an additional metal.

4. The medical device according to claim 1, wherein said surface layer is a metallic layer and said compound of a non-toxic post-transition metal is a metallic compound.

5. The medical device according to claim 1, wherein said non-toxic post-transition metal is selected from bismuth and gallium.

6. The medical device according to claim 1, wherein said compound of a non-toxic post-transition metal is selected from the group consisting of gallium-titanium, gallium-titanium oxide, gallium-titanium nitride, gallium nitride, gallium carbide, gallium selenide, and gallium sulphide, gallium chloride, gallium fluoride, gallium iodide, gallium oxalate, gallium phosphate, gallium maltolate, gallium acetate and gallium lactate.

7. The medical device according to claim 1, wherein said compound of a non-toxic post-transition metal is selected from the group consisting of bismuth-titanium, bismuth-titanium oxide, bismuth-titanium nitride, bismuth nitride, bismuth carbide, bismuth selenide, and bismuth sulphide, bismuth chloride, bismuth fluoride, bismuth iodide, bismuth oxalate, bismuth phosphate, bismuth maltolate, bismuth acetate and bismuth lactate.

8. The medical device according to claim 1, wherein said compound of a non-toxic post-transition metal is a nitride.

9. The medical device according to claim 8, wherein said nitride is selected from the group consisting of gallium-titanium nitride, gallium nitride, bismuth-titanium nitride, bismuth nitride, and gallium-bismuth-titanium nitride.

10. The medical device according to claim 1, wherein said compound of a non-toxic post-transition metal constitutes the major part of said layer.

11. The medical device according to claim 1, further comprising a salt of a non-toxic post-transition metal deposited onto said surface layer.

12. The medical device according to claim 1, wherein said surface layer has a thickness in the range of from 10 nm to 1.5 μm.

13. The medical device according to claim 1, wherein said layer is a homogeneous layer.

14. The medical device according to claim 1, wherein said layer is a non-porous layer.

15. The medical device according to claim 1, wherein said substrate comprises a metallic material.

16. The medical device according to claim 1, wherein said substrate comprises a ceramic material.

17. The medical device according to claim 1, wherein said substrate comprises a polymeric material.

18. The medical device according to claim 1, wherein said substrate comprises a composite material.

19. The medical device according to any one of the preceding claims, which is an implant intended for long-term contact with living tissue.

20. The medical device according to claim 1, which is intended for prolonged contact with living tissue.

21. The medical device according to claim 1, which is intended for short-term contact with living tissue.

22. The medical device according to claim 1, which is a dental implant.

23. The medical device according to claim 22, wherein said dental implant is a dental abutment.

24. The medical device according to claim 1, which is a bone anchored hearing device.

25. The medical device according to claim 1, which is a stent.

26. The medical device according to claim 1, which is a shunt.

27. The medical device according to any one of the claims 1 to 20, which is an orthopaedic implant.

28. The medical device according to claim 1, which is a catheter for insertion into a bodily cavity.

29. A method of producing a medical device according to claim 1 comprising steps of:
a) providing a substrate having a surface; and
b) applying a compound of a non-toxic post-transition metal onto said surface to form a layer.

30. The method according to claim 29, wherein said compound of a non-toxic post-transition metal is a nitride of a non-toxic post-transition metal.

31. The method according to claim 29, wherein step b) involves applying a non-toxic post-transition metal and an additional metal or metal compound onto said surface.

32. The method according to claim 31, wherein said additional metal or metal compound comprises titanium.

33. The method according to claim 29, wherein step b) is performed using a thin film deposition technique.
